(19) United States
(12) Patent Application Publication
(10) Pub. No.: US 2024/0315953 A1
(43) Pub. Date: Sep. 26, 2024

(54) HAIR CARE COMPOSITION AND METHODS OF PREPARING THE SAME AND PREPARING PLANT EXTRACTS

(71) Applicant: Ameka COLEMAN, Brandon, MS (US)

(72) Inventor: Ameka COLEMAN, Brandon, MS (US)

(21) Appl. No.: 18/439,324

(22) Filed: Feb. 12, 2024

Related U.S. Application Data
(60) Provisional application No. 63/454,577, filed on Mar. 24, 2023.

(51) Int. Cl. A61K 8/9789; A61Q 5/06

(52) U.S. Cl. CPC A61K 8/9789; A61Q 5/06; A61K 2800/52

(57) ABSTRACT

Hair care compositions, methods of use and methods of preparing the same are provided. The hair care compositions can include a blend of six plant extracts including teasel root extract, ashwaganda leaf extract, rosemary leaf extract, mulberry leaf extract, malva leaf extract and Echinacea purpurea root extract. The hair care compositions can be used to soothe and stimulate the scalp and/or hair edge follicles.

### CROSS-REFERENCE TO RELATED APPLICATIONS

This application claims the benefit of, and priority to, U.S. Provisional Patent Application No. 63/454,577, filed Mar. 24, 2023, which is incorporated by reference herein in its entirety.

### FIELD

The present disclosure relates to hair care compositions and, in particular, to a multi-benefit product for textured hair that acts as a salve and pomade, for the treatment of the scalp which may relieve eczema and/or psoriasis, soothe and stimulate the scalp and/or hair edge follicles, decrease dryness of the hair and/or scalp, improve blood circulation to the scalp, prevent breakage, prevent dandruff, and/or reduce antimicrobial activity of the scalp which, in turn, in some aspects, can help facilitate length retention and/or reduction in shedding, and to methods of making the same.

### BACKGROUND

Textured hair has unique and complex needs that have often been overlooked in the hair care industry when it comes to providing quality and effective products that aren't filled with harsh chemicals. For many years, hair products on the market were not specifically formulated for textured hair and had harsh chemicals that left the hair feeling dry and brittle which eventually leads to breakage. While there are now more options available for textured hair people, there is still a great need for a new scalp health and retention product that is specifically designed for the needs of those with textured hair and that provides the wonderful benefits of soothing and healing ingredients in effective amounts.

Textured hair is naturally drier than straight hair because the natural oils produced by the scalp have a harder time traveling down the hair shaft due to spiral nature. Salves and pomades are two types of products that can positively benefit textured hair but they also come with several limitations and need to be improved upon. Salves have been used for centuries to treat various skin conditions. While they can be effective in providing temporary relief and promoting healing, they also have their limitations. Many salves are thick and oily leading to a greasy or oily residue on the skin. Furthermore, existing salves are not specifically formulated for use on the hair and scalp of textured hair, which have unique needs and challenges.

Pomades have been a popular hair styling product for decades, providing a sleek finished look. However, they also have limitations specifically when it comes to the hair and scalp. Many pomades contain heavy and harsh ingredients like petroleum jelly and mineral oil. These ingredients work by blocking moisture from penetrating the scalp. This causes a clog in the pores which leads to breakage of the hair, dryness, and scalp irritation. It can also lead to hair loss because the follicles become damaged and unable to produce new healthy strands. While some pomades may include healing ingredients like natural oils, they are often present in low concentrations and do not provide sufficient nourishment for the hair and scalp, which is needed significantly more in textured hair.

Another limitation of traditional pomades is their lack of intentional focus on scalp health. Many of the current hair styling products are designed solely to provide a superficial end result like hold and shine, without any consideration for the health of the scalp. However, a healthy scalp is essential for healthy hair growth, and neglective it can lead to a range of issues such as dandruff, scalp acne, and hair loss.

As a result, there is a need for a new salve and pomade product that is specifically designed to address the unique needs of textured hair and scalp, providing effective healing and nourishment, while also promoting healthy hair growth and good scalp health, without the drawbacks of traditional salves and pomades ingredients.

### SUMMARY

The present disclosure presents a hair and scalp composition that effectively addresses common hair problems such as dryness, breakage, and shedding. The carefully selected active ingredients work in synergy to promote hair growth and length retention, while simultaneously treating a wide range of skin conditions such as psoriasis and eczema. The formulation is specifically designed for textured hair types and includes ingredients that soothe and stimulate the scalp and hair edge follicles, leading to enhanced absorption and maximum effectiveness. The unique combination of active ingredients in the present disclosure has not been previously disclosed. Overall, this salve and pomade hair composition provides a safe and effective solution for maintaining healthy, beautiful hair while also providing therapeutic and healing benefits to the scalp. The formulation also includes ingredients that reduce antimicrobial activity of the scalp, which can further aid in length retention, reduction of breakage, and decreasing dryness. Moreover, this hair composition holds the hair in place, providing styling benefits, while also promoting the overall health of the hair and scalp.

In some embodiments, a hair care composition is provided comprising from about 0.5% to about 20% (w/w) teasel root extract; and one or more additional extracts selected from the group consisting of ashwagandha leaf extract, rosemary leaf extract, mulberry leaf extract, malva leaf extract, Echinacea purpurea root extract, and combinations thereof, wherein each of the one or more additional extracts is present in an amount from about 0.5% to about 15% (w/w) based on the total weight of the composition.

In some embodiments, a method for styling or sculpting hair is provided, comprising applying a hair care composition of the present disclosure to hair.

In some embodiments, a method is provided for producing a plant extract, comprising: combining a plant material with extractant containing glycerin and propanediol and allowing the plant material to be extracted for a period of time to yield a plant extract.

In some embodiments, A method for producing a hair care composition, comprising: combining a teasel root extract and optionally, an ashwagandha leaf extract, a rosemary leaf extract, a mulberry leaf extract, a malva leaf extract, and an Echinacea purpurea root extract, water, an humectant and, optionally, a stabilizer to yield an extract formulation; combining the extract formulation with a formulation base; mixing the extract formulation and formulation base to yield a mixed extract and formulation base; combining the mixed extract formulation and formulation base with additional ingredients; and allowing the mixed extract formulation, formulation base and additional ingredients to solidify to yield the hair care composition

### BRIEF DESCRIPTION OF THE DRAWINGS

FIG. 1 shows before and after images of a human subject who used a hair care composition of the present composition to treat postpartum shedding.

### DETAILED DESCRIPTION

### Definitions

Unless otherwise defined, all terms of art, notations and other scientific terminology used herein are intended to have the meanings commonly understood by those of skill in the art to which this disclosure. In some cases, terms with commonly understood meanings are defined herein for clarity and/or for ready reference. The inclusion of such definitions herein should not necessarily be construed to represent a difference over what is generally understood in the art. The techniques and procedures described or referenced herein are generally well understood and commonly employed using conventional methodologies by those skilled in the art.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value +/−10%, +/−5%, or +/−1%.

The term “combinations thereof” includes every possible combination of elements to which the term refers to.

As used herein, “textured hair” refers to hair that comprises strands that have shape such as, by way of example but not limitation, a curl pattern. Exemplary types of textured hair include type 2 (wavy), type 3 (curly), and type 4 (coily).

As used herein, “fragrance” refers to any natural or synthetic substance used solely to impart an odor to a composition of the present disclosure.

As used herein, “propanediol” refers to 1,2-propanediol or 1,3-propanediol. In certain embodiments, the propanediol is 1,3-propaneidol. In certain embodiments, the propanediol is 1,2-propanediol. In certain embodiments, the propanediol is a mixture of 1,2-propanediol and 1,3-propanediol.

An issue with mainstream textured hair products is that the healing extracts are usually not present in meaningful amounts that will produce the best results for the end user. Additionally, many of the harsh ingredients in the hair products, such as pomades, mask any of the previous low amount of the beneficial ingredients. The hair care compositions of the present disclosure overcome these issues by combining powerful healing extracts in combinations and amounts that will provide effective therapeutic treatment and results. Overall, the present disclosure provides new and unique combinations of specifically and specially selected leaf and root extracts.

The hair care compositions of the present disclosure can include a blend of extracts which can include teasel root extract, ashwagandha leaf extract, rosemary leaf extract, mulberry leaf extract, malva leaf extract and/or purple coneflower root extract. Additional extracts, such as apricot extract or fenugreek seed extract, can be combined with these extracts. In certain aspects, an additive for a hair care composition can include the blend of teasel root (Dipsacus fullonum) extract, ashwagandha leaf (Withania somnifera) extract, rosemary leaf (Rosemarinus officinalis) extract, mulberry leaf (Morus alba) extract, malva leaf (Malva sylvestris) extract and purple coneflower (Echinacea purpurea) root extract.

Teasel root (Dipsacus fullonum) is known for its antimicrobial properties, which can potentially prevent scalp infections and promote optimal scalp health. This extract is also anti-inflammatory and soothing which helps conditions such as eczema and psoriasis that affect the scalp. It may also provide nutrients to the hair and moisturize its texture, thereby reducing susceptibility to breakage and damage.

Ashwaganda leaf (Withania somnifera) is native to Asia and Africa and possesses anti-inflammatory properties which can help soothe dry and itchy scalps.

Rosemary leaf (Rosemarinus officinalis) possesses antimicrobial and anti-inflammatory properties that can help to stimulate hair growth while also preventing hair loss.

Mulberry leaf (Morus alba) is full of antioxidants, vitamins and minerals that can nourish the hair and scalp.

Malva leaf (Malva sylvestris) is a natural emollient that can hydrate and soothe dry and irritated scalp.

Purple coneflower (Echinacea purpurea) root possesses antimicrobial and anti-inflammatory properties that can help promote good scalp health.

It has surprisingly been found that a hair care composition that includes a teasel root extract has improved benefits compared to the use of the other extracts alone.

In some embodiments, a hair care composition is provided that comprises teasel root extract.

In some embodiments, a hair care composition is provided that comprises teasel root extract and one or more additional extracts. In certain embodiments, the one or more additional extracts are selected from the group consisting of ashwaganda leaf extract, rosemary leaf extract, mulberry leaf extract, malva leaf extract and Echinacea purpurea root extract. In certain embodiments, the one or more additional extracts are selected from the group consisting of ashwaganda leaf extract, rosemary leaf extract, mulberry leaf extract, malva leaf extract, Echinacea purpurea root extract, apricot extract and fenugreek seed extract. In some embodiments, a hair care composition is provided that comprises teasel root extract and one, two, three, four, five, six, seven or more additional extracts. In certain embodiments, the additional extracts are selected from the group consisting of ashwagandha leaf extract, rosemary leaf extract, mulberry leaf extract, malva leaf extract and Echinacea purpurea root extract. In certain embodiments, the additional extracts are selected from the group consisting of ashwagandha leaf extract, rosemary leaf extract, mulberry leaf extract, malva leaf extract, Echinacea purpurea root extract, apricot extract and fenugreek seed extract. In certain embodiments, a combination of extracts in the hair care composition consists of consists essentially of the teasel root extract and the additional extracts disclosed herein. In certain embodiments, the combination of extracts in the hair care composition consists or consists essentially of the teasel root extract and the additional extracts disclosed herein.

In certain embodiments, the hair care composition comprises from about 1% to about 30% (w/w) of the teasel root extract and additional extracts. In certain embodiments, the hair care composition comprises from about 1% to about 30% (w/w), about 1% to about 25% (w/w), about 1% to about 20% (w/w), about 1% to about 15% (w/w), about 1% to about 10% (w/w), about 1% to about 5% (w/w), about 1% to about 4% (w/w), about 1% to about 3% (w/w), about 1% to about 2% (w/w), about 2% to about 30% (w/w), about 2% to about 25% (w/w), about 2% to about 20% (w/w), about 2% to about 15% (w/w), about 2% to about 10% (w/w) about 2% to about 5% (w/w), about 2% to about 4% (w/w), about 2% to about 3% (w/w), about 3% to about 30% (w/w), about 3% to about 25% (w/w), about 3% to about 20% (w/w), about 3% to about 15% (w/w), about 3% to about 10% (w/w), about 3% to about 5% (w/w), about 3% to about 4% (w/w), about 4% to about 30% (w/w), about 4% to about 25% (w/w), about 4% to about 20% (w/w), about 4% to about 15% (w/w), about 4% to about 10% (w/w), about 4% to about 5% (w/w), about 5% to about 30% (w/w), about 5% to about 25% (w/w), about 5% to about 20% (w/w), about 5% to about 15% (w/w), about 5% to about 10% (w/w), about 10% to about 30% (w/w), about 10% to about 25% (w/w), about 10% to about 20% (w/w), about 10% to about 15% (w/w), about 15% to about 30% (w/w), about 15% to about 25% (w/w), about 15% to about 20% (w/w), about 20% to about 30% (w/w), about 20% to about 25% (w/w), about 25% to about 30% (w/w), about 1%, 2%, 3%, 4%, 5%, 10%, 15%, 20%, 25%, or 30% (w/w) of the teasel root extract and additional extracts.

In some embodiments, a hair care composition is provided that comprises teasel root extract, ashwaganda leaf extract, rosemary leaf extract, mulberry leaf extract, malva leaf extract and Echinacea purpurea root extract. In certain embodiments, the hair composition comprises from about 0.5% to about 20% (w/w) teasel root extract, from about 0.5% to about 15% (w/w) ashwagandha leaf extract, from about 0.5% to about 15% (w/w) rosemary leaf extract, from about 0.5% to about 15% (w/w) mulberry leaf extract, from about 0.5% to about 15% (w/w) malva leaf extract, and from about 0.5% to about 15% (w/w) Echinacea purpurea root extract based on the total weight of the hair care composition.

In certain embodiments, the teasel root extract can be present in an amount from about 0.5% to about 20% (w/w) based on the total weight of the hair care composition. In certain embodiments, the teasel root extract can be present in an amount from about 0.5% to about 20% (w/w), about 0.5% to about 15% (w/w), about 0.5% to about 10% (w/w), about 0.5% to about 5% (w/w), about 0.5% to about 1% (w/w), about 1% to about 20% (w/w), about 1% to about 15% (w/w), about 1% to about 10% (w/w), about 1% to about 5% (w/w), about 5% to about 20% (w/w), about 5% to about 15% (w/w), about 5% to about 10% (w/w), about 10% to about 20% (w/w), about 10% to about 15% (w/w), about 15% to about 20% (w/w), or about 0.5%, 0.75%, 1%, 1.5%, 2%, 2.5%, 3%, 3.5%, 4%, 5%, 7.5%, 10%, 12.5%, 15%, 17.5% or 20% (w/w) based on the total weight of the hair care composition. In certain embodiments, the teasel root extract can be present in an amount of about 0.5% (w/w) based on the total weight of the hair care composition. In certain embodiments, the teasel root extract can be present in an amount from about 15% to about 20% (w/w) based on the total weight of the hair care composition. In certain embodiments, the teasel root extract can be present in an amount from about 14% to about 18% (w/w) based on the total weight of the hair care composition. In certain embodiments, the teasel root extract can be present in an amount from about 12% to about 15% (w/w) based on the total weight of the hair care composition.

In certain embodiments, the ashwagandha leaf extract can be present in an amount from about 0.5% to about 15% (w/w) based on the total weight of the hair care composition. In certain embodiments, the ashwagandha leaf extract can be present in an amount from about 0.5% to about 15% (w/w), about 0.5% to about 10% (w/w), about 0.5% to about 5% (w/w), about 0.5% to about 1% (w/w), about 1% to about 15% (w/w), about 1% to about 10% (w/w), about 1% to about 5% (w/w), about 5% to about 15% (w/w), about 5% to about 10% (w/w), about 10% to about 15% (w/w), or about 0.5%, 0.75%, 1%, 1.5%, 2%, 2.5%, 3%, 3.5%, 4%, 4.5%, 5%, 7.5%, 10%, 12.5% or 15% (w/w) based on the total weight of the hair care composition. In certain embodiments, the ashwagandha leaf extract can be present in an amount of about 0.5% (w/w) based on the total weight of the hair care composition.

In certain embodiments, the rosemary leaf extract can be present in an amount from about 0.5% to about 15% (w/w) based on the total weight of the hair care composition. In certain embodiments, the rosemary leaf extract can be present in an amount from about 0.5% to about 15% (w/w), about 0.5% to about 10% (w/w), about 0.5% to about 5% (w/w), about 0.5% to about 1% (w/w), about 1% to about 15% (w/w), about 1% to about 10% (w/w), about 1% to about 5% (w/w), about 5% to about 15% (w/w), about 5% to about 10% (w/w), about 10% to about 15% (w/w), or about 0.5%, 0.75%, 1%, 1.5%, 2%, 2.5%, 3%, 3.5%, 4%, 4.5%, 5%, 7.5%, 10%, 12.5% or 15% (w/w) based on the total weight of the hair care composition. In certain embodiments, the rosemary leaf extract can be present in an amount of about 0.5% (w/w) based on the total weight of the hair care composition.

In certain embodiments, the mulberry leaf extract can be present in an amount from about 0.5% to about 15% (w/w) based on the total weight of the hair care composition. In certain embodiments, the mulberry leaf extract can be present in an amount from about 0.5% to about 15% (w/w), about 0.5% to about 10% (w/w), about 0.5% to about 5% (w/w), about 0.5% to about 1% (w/w), about 1% to about 15% (w/w), about 1% to about 10% (w/w), about 1% to about 5% (w/w), about 5% to about 15% (w/w), about 5% to about 10% (w/w), about 10% to about 15% (w/w), or about 0.5%, 0.75%, 1%, 1.5%, 2%, 2.5%, 3%, 3.5%, 4%, 4.5%, 5%, 7.5%, 10%, 12.5% or 15% (w/w) based on the total weight of the hair care composition. In certain embodiments, the mulberry leaf extract can be present in an amount of about 0.5% (w/w) based on the total weight of the hair care composition.

In certain embodiments, the malva leaf extract can be present in an amount from about 0.5% to about 15% (w/w) based on the total weight of the hair care composition. In certain embodiments, the malva leaf extract can be present in an amount from about 0.5% to about 15% (w/w), about 0.5% to about 10% (w/w), about 0.5% to about 5% (w/w), about 0.5% to about 1% (w/w), about 1% to about 15% (w/w), about 1% to about 10% (w/w), about 1% to about 5% (w/w), about 5% to about 15% (w/w), about 5% to about 10% (w/w), about 10% to about 15% (w/w), or about 0.5%, 0.75%, 1%, 1.5%, 2%, 2.5%, 3%, 3.5%, 4%, 4.5%, 5%, 7.5%, 10%, 12.5% or 15% (w/w) based on the total weight of the hair care composition. In certain embodiments, the malva leaf extract can be present in an amount of about 0.5% (w/w) based on the total weight of the hair care composition.

In certain embodiments, the Echinacea purpurea root extract can be present in an amount from about 0.5% to about 15% (w/w) based on the total weight of the hair care composition. In certain embodiments, the Echinacea purpurea root extract can be present in an amount from about 0.5% to about 15% (w/w), about 0.5% to about 10% (w/w), about 0.5% to about 5% (w/w), about 0.5% to about 1% (w/w), about 1% to about 15% (w/w), about 1% to about 10% (w/w), about 1% to about 5% (w/w), about 5% to about 15% (w/w), about 5% to about 10% (w/w), about 10% to about 15% (w/w), or about 0.5%, 0.75%, 1%, 1.5%, 2%, 2.5%, 3%, 3.5%, 4%, 4.5%, 5%, 7.5%, 10%, 12.5% or 15% (w/w) based on the total weight of the hair care composition. In certain embodiments, the Echinacea purpurea root extract can be present in an amount of about 0.5% (w/w) based on the total weight of the hair care composition.

In certain embodiments, the hair care composition can further comprise additional plant extracts. In certain embodiments, the additional plant extracts are selected from the group consisting of apricot extract, fenugreek seed extract, and combinations thereof. In certain embodiments, the apricot extract is present in an amount from about 0.5% to about 5% (w/w) based on the total weight of the hair care composition. In certain embodiments, the apricot extract is present in an amount from about 0.5% to about 1% (w/w), about 0.5% to about 0.8%, or about 0.3% to about 0.7% (w/w) based on the total weight of the hair care composition. In certain embodiments, the fenugreek seed extract is present in an amount from about 0.5% to about 5% (w/w) based on the total weight of the hair care composition. In certain embodiments, the fenugreek seed extract is present in an amount from about 0.5% to about 1% (w/w), about 0.5% to about 0.8% (w/w), or about 0.3% to about 0.7% (w/w) based on the total weight of the hair care composition. In certain embodiments, the apricot extract is an apricot oil.

In certain embodiments, the hair care composition can further comprise water in an amount from about 30% to about 70% (w/w) based on the total weight of the hair care composition. In certain embodiments, the hair care composition can further comprise water in an amount from about 40% to about 70% (w/w) based on the total weight of the hair care composition. In certain embodiments, the hair care composition can further comprise water in an amount from about 30% to about 60% (w/w), about 30% to about 50% (w/w), about 30% to about 40% (w/w), about 40% to about 70% (w/w), about 40% to about 60% (w/w), about 40% to about 50% (w/w), about 50% to about 70% (w/w), about 50% to about 60% (w/w), or about 30%, 35%, 40%, 45%, 50%, 55%, 60%, 65%, or 70% (w/w) based on the total weight of the hair care composition. In certain embodiments, water can be present in an amount sufficient to constitute the balance of the composition.

In certain embodiments, the hair care composition further comprises a shaper or holder, an emollient and/or a humectant.

In certain embodiments, the shaper (holder) can be present in an amount from about 10% to about 50% (w/w) based on the total weight of the hair care composition. In certain embodiments, the shaper can be present in an amount from about 10% to about 50% (w/w), about 10% to about 45% (w/w), about 10% to about 40% (w/w), about 10% to about 35% (w/w), about 10% to about 30% (w/w), about 10% to about 25% (w/w), about 10% to about 20% (w/w), about 10% to about 15% (w/w), about 15% to about 50% (w/w), about 15% to about 45% (w/w), about 15% to about 40% (w/w), about 15% to about 35% (w/w), about 15% to about 30% (w/w), about 15% to about 25% (w/w), about 15% to about 20% (w/w), about 20% to about 50% (w/w), about 20% to about 45% (w/w), about 20% to about 40% (w/w), about 20% to about 35% (w/w), about 20% to about 30% (w/w), about 20% to about 25% (w/w), about 25% to about 50% (w/w), about 25% to about 45% (w/w), about 25% to about 40% (w/w), about 25% to about 35% (w/w), about 25% to about 30% (w/w), about 30% to about 50% (w/w), about 30% to about 45% (w/w), about 30% to about 40% (w/w), about 30% to about 35% (w/w), about 35% to about 50% (w/w), about 35% to about 45% (w/w), about 35% to about 40% (w/w), about 40% to about 50% (w/w), about 40% to about 45% (w/w), about 45% to about 50% (w/w), or about 10%, 15%, 20%, 25%, 30%, 35%, 40%, 45%, or 50% (w/w) based on the total weight of the hair care composition. In certain embodiments, the shaper is selected from the group consisting of maltodextrin, Ceteareth-25, and combinations thereof. In certain embodiments, the hair care composition further comprises Ceteareth-25. In certain embodiments, the hair care composition further comprises maltodextrin. In certain embodiments, the hair care composition comprises Ceteareth-25 and maltodextrin. In certain embodiments, Ceteareth-25 is present in an amount from about 10% to about 30% (w/w) based on the total weight of the hair care composition. In certain embodiments, Ceteareth-25 is present in an amount from about 10% to about 30% (w/w), about 10% to about 25% (w/w), about 10% to about 20% (w/w), about 10% to about 15% (w/w), about 15% to about 30% (w/w), about 15% to about 25% (w/w), about 15% to about 20% (w/w), about 20% to about 30% (w/w), about 20% to about 25% (w/w), about 25% to about 30% (w/w), or about 10%, 15%, 20%, 25%, or 30% (w/w) based on the total weight of the hair care composition. In certain embodiments, Ceteareth-25 is present in an amount from about 12% to about 16% (w/w) based on the total weight of the hair care composition. In certain embodiments, Ceteareth-25 is present in an amount from about 14% to about 18% (w/w) based on the total weight of the hair care composition. In certain embodiments, Ceteareth-25 is present in an amount from about 19% to about 25% (w/w) based on the total weight of the hair care composition. In certain embodiments, Ceteareth-25 is present in an amount of about 10% (w/w) based on the total weight of the hair care composition. In certain embodiments, Ceteareth-25 is present in an amount of about 15% (w/w) based on the total weight of the hair care composition. In certain embodiments, Ceteareth-25 is present in an amount of about 20% (w/w) based on the total weight of the hair care composition. In certain embodiments, Ceteareth-25 is present in an amount of about 25% (w/w) based on the total weight of the hair care composition. In certain embodiments, Ceteareth-25 is present in an amount of about 30% (w/w) based on the total weight of the hair care composition. In certain embodiments, the hair care composition further comprises maltodextrin. In certain embodiments, maltodextrin is present in an amount from about 5% to about 25% (w/w) based on the total weight of the hair care composition. In certain embodiments, maltodextrin is present in an amount from about 5% to about 25% (w/w), about 5% to about 20% (w/w), about 5% to about 15% (w/w), about 5% to about 10% (w/w), about 10% to about 25% (w/w), about 10% to about 20% (w/w), about 10% to about 15% (w/w), about 15% to about 25% (w/w), about 15% to about 20% (w/w), about 20% to about 25% (w/w), or about 5%, 10%, 15%, 20%, or 25% (w/w) based on the total weight of the hair care composition. In certain embodiments, maltodextrin is present in an amount from about 20% to about 25% (w/w) based on the total weight of the hair care composition. In certain embodiments, maltodextrin is present in an amount from about 15% to about 19% (w/w) based on the total weight of the hair care composition. In certain embodiments, maltodextrin is present in an amount from about 10% to about 14% (w/w) based on the total weight of the hair care composition. In certain embodiments, maltodextrin is present in an amount of about 5% (w/w) based on the total weight of the hair care composition. In certain embodiments, maltodextrin is present in an amount of about 10% (w/w) based on the total weight of the hair care composition. In certain embodiments, maltodextrin is present in an amount of about 15% (w/w) based on the total weight of the hair care composition. In certain embodiments, maltodextrin is present in an amount of about 20% (w/w) based on the total weight of the hair care composition. In certain embodiments, maltodextrin is present in an amount of about 25% (w/w) based on the total weight of the hair care composition.

In certain embodiments, the emollient can be present in an amount from about 1% to about 5% (w/w) based on the total weight of the hair care composition. In certain embodiments, the emollient can be present in an amount from about 1% to about 5% (w/w), about 1% to about 4% (w/w), about 1% to about 3% (w/w), about 1% to about 2% (w/w), about 2% to about 5% (w/w), about 2% to about 4% (w/w), about 2% to about 3% (w/w), about 3% to about 5% (w/w), about 3% to about 4% (w/w), about 4% to about 5% (w/w), or about 1%, 2%, 3%, 4%, or 5% (w/w) based on the total weight of the hair care composition. In certain embodiments, the emollient is selected from the group consisting of PEG-7 glyceryl cocoate, PEG 40 hydrogenated castor oil, and combinations thereof. In certain embodiments, the hair care composition further comprises PEG-7 glyceryl cocoate. In certain embodiments, the hair care composition further comprises PEG 40 hydrogenated castor oil. In certain embodiments, the hair care composition further comprises PEG-7 glyceryl cocoate and PEG 40 hydrogenated castor oil. In certain embodiments, PEG-7 glyceryl cocoate is present in an amount from about 0.6% to about 3% (w/w) based on the total weight of the hair care composition. In certain embodiments, PEG-7 glyceryl cocoate is present in an amount of about 0.6% (w/w) based on the total weight of the hair care composition. In certain embodiments, PEG-7 glyceryl cocoate is present in an amount of about 1% (w/w) based on the total weight of the hair care composition. In certain embodiments, PEG-7 glyceryl cocoate is present in an amount of about 1.5% (w/w) based on the total weight of the hair care composition. In certain embodiments, PEG-7 glyceryl cocoate is present in an amount of about 2% (w/w) based on the total weight of the hair care composition. In certain embodiments, PEG-7 glyceryl cocoate is present in an amount of about 2.5% (w/w) based on the total weight of the hair care composition. In certain embodiments, PEG-7 glyceryl cocoate is present in an amount of about 3% (w/w) based on the total weight of the hair care composition. In certain embodiments, PEG 40 hydrogenated castor oil is present in an amount from about 0.5% to about 2% (w/w) based on the total weight of the hair care composition. In certain embodiments, PEG 40 hydrogenated castor oil is present in an amount from about 0.5% to about 2% (w/w), about 0.5% to about 1.5% (w/w), about 0.5% to about 1% (w/w), about 1% to about 2% (w/w), about 1% to about 1.5% (w/w), about 1.5% to about 2% (w/w), or about 0.5%, 1%, 1.5%, or 2% (w/w) based on the total weight of the hair care composition. In certain embodiments, PEG 40 hydrogenated castor oil is present in an amount of about 0.5% (w/w) based on the total weight of the hair care composition. In certain embodiments, PEG 40 hydrogenated castor oil is present in an amount of about 1% (w/w) based on the total weight of the hair care composition. In certain embodiments, PEG 40 hydrogenated castor oil is present in an amount of about 1.5% (w/w) based on the total weight of the hair care composition. In certain embodiments, PEG 40 hydrogenated castor oil is present in an amount of about 2% (w/w) based on the total weight of the hair care composition.

In certain embodiments, the humectant can be present in an amount from about 3% to about 25% (w/w) based on the total weight of the hair care composition. In certain embodiments, the humectant can be present in an amount from about 3% to about 25% (w/w), about 3% to about 20% (w/w), about 3% to about 15% (w/w), about 3% to about 10% (w/w), about 3% to about 5% (w/w), about 5% to about 25% (w/w), about 5% to about 20% (w/w), about 5% to about 15% (w/w), about 5% to about 10% (w/w), about 10% to about 25% (w/w), about 10% about 20% (w/w), about 10% to about 15% (w/w), about 15% to about 25% (w/w), about 15% to about 20% (w/w), about 20% to about 25% (w/w), or about 3%, 4%, 5%, 6%, 7%, 8%, 9%, 10%, 11%, 12%, 13%, 14%, 15%, 20%, or 25% (w/w) based on the total weight of the hair care composition. In certain embodiments, the humectant is selected from the group consisting of glycerin, propanediol, and combinations thereof. In certain embodiments, the hair care composition further comprises glycerin. In certain embodiments, the hair care composition further comprises propanediol. In certain embodiments, the hair care composition further comprises glycerin and propanediol. In certain embodiments, glycerin is present in an amount from about 2% to about 15% (w/w) based on the total weight of the hair care composition. In certain embodiments, glycerin is present in an amount from about 2% to about 15% (w/w), about 2% to about 10% (w/w), about 2% to about 5% (w/w), about 5% to about 15% (w/w), about 5% to about 10% (w/w), about 10% to about 15% (w/w), or about 2%, 3%, 4%, 5%, 6%, 7%, 8%, 9%, 10%, 11%, 12%, 13%, 14%, or 15% (w/w) based on the total weight of the hair care composition. In certain embodiments, propanediol is present in an amount of about 0.01% to about 15% (w/w) based on the total weight of the hair care composition. In certain embodiments, propanediol is present in an amount from about 2% to about 15% (w/w) based on the total weight of the hair care composition. In certain embodiments, propanediol is present in an amount from about 0.01% to about 15% (w/w), about 0.01% to about 10% (w/w), about 0.01% to about 5% (w/w), about 0.01% to about 2% (w/w), about 0.01% to about 1% (w/w), about 0.05% to about 15% (w/w), about 0.05% to about 10% (w/w), about 0.05% to about 5% (w/w), about 0.05% to about 2% (w/w), about 0.05% to about 1% (w/w), about 0.1% to about 15% (w/w), about 0.1% to about 10% (w/w), about 0.1% to about 5% (w/w), about 0.1% to about 2% (w/w), about 0.1% to about 1% (w/w), about 0.5% to about 15% (w/w), 0.5% to about 10% (w/w), about 0.5% to about 5% (w/w), about 0.5% to about 2% (w/w), about 0.5% to about 1% (w/w), about 1% to about 15% (w/w), about 1% to about 10% (w/w), about 1% to about 5% (w/w), about 1% to about 2% (w/w), about 2% to about 15% (w/w), about 2% to about 10% (w/w), about 2% to about 5% (w/w), about 5% to about 15% (w/w), about 5% to about 10% (w/w), about 10% to about 15% (w/w), or about 0.01%, 0.05%, 0.1%, 0.5%, 1%, 2%, 3%, 4%, 5%, 6%, 7%, 8%, 9%, 10%, 11%, 12%, 13%, 14%, or 15% (w/w) based on the total weight of the hair care composition.

In certain embodiments, the hair care composition can further comprise one or more ingredients selected from the group consisting of glycerin, propanediol, PEG 40 hydrogenated castor oil, PEG-7 glyceryl cocoate, Ceteareth-25, tetrasodium glutamate diacetate and maltodextrin. In certain embodiments, the hair care composition further comprises glycerin, propanediol, PEG 40 hydrogenated castor oil, PEG-7 glyceryl cocoate, Ceteareth-25 and maltodextrin as described herein. In certain embodiments, the hair care composition further comprises glycerin, propanediol, PEG 40 hydrogenated castor oil, PEG-7 glyceryl cocoate, Ceteareth-25, tetrasodium glutamate diacetate and maltodextrin as described herein.

In certain embodiments, the hair care composition can further comprise a stabilizer. In certain embodiments, the stabilizer is selected from the group consisting of tetrasodium glutamate diacetate, tetrasodium EDTA, and combinations thereof. In certain embodiments, the stabilizer comprises tetrasodium glutamate diacetate (TGD). In certain embodiments, the stabilizer comprises tetrasodium EDTA. In certain embodiments, the stabilizer is present in an amount from about 0.25% to about 1% (w/w) based on the total weight of the hair care composition. In certain embodiments, the stabilizer is present in an amount from about 0.25% to about 1% (w/w), about 0.25% to about 0.75% (w/w), about 0.25% to about 0.5% (w/w), about 0.5% to about 0.75% (w/w), or about 0.25%, 0.3%, 0.4%, 0.5%, 0.6%, 0.7%, 0.75%, 0.8%, 0.9%, or 1% (w/w) based on the total weight of the hair care composition. In certain embodiments, tetrasodium glutamate diacetate is present in an amount from about 0.25% to about 1% (w/w) based on the total weight of the hair care composition. In certain embodiments, tetrasodium glutamate diacetate is present in an amount from about 0.25% to about 1% (w/w), about 0.25% to about 0.75% (w/w), about 0.25% to about 0.5% (w/w), about 0.5% to about 0.75% (w/w), or about 0.25%, 0.3%, 0.4%, 0.5%, 0.6%, 0.7%, 0.75%, 0.8%, 0.9%, or 1% (w/w) based on the total weight of the hair care composition. In certain embodiments, tetrasodium EDTA is present in an amount from about 0.25% to about 1% (w/w), about 0.25% to about 0.75% (w/w) based on the total weight of the hair care composition. In certain embodiments, tetrasodium EDTA is present in an amount from about 0.25% to about 0.75% (w/w), about 0.25% to about 0.5% (w/w), about 0.5% to about 0.75% (w/w), or about 0.25%, 0.3%, 0.4%, 0.5%, 0.6%, 0.7%, 0.75%, 0.8%, 0.9%, or 1% (w/w) based on the total weight of the hair care composition.

In certain embodiments, the hair care composition further comprises a preservative. In certain embodiments, the preservative can be present in an amount from about 0.01% to about 2% (w/w) based on the total weight of the hair care composition. In certain embodiments, the preservative can be present in an amount from about 0.01% to about 2% (w/w), about 0.01% to about 1.5% (w/w), about 0.01% to about 1% (w/w), about 0.01% to about 0.5% (w/w), about 0.01% to about 0.1% (w/w), about 0.1% to about 2% (w/w), about 0.1% to about 1.5% (w/w), about 0.1% to about 1% (w/w), about 0.1% to about 0.5% (w/w), about 0.2% to about 2% (w/w), about 0.2% to about 1.5% (w/w), about 0.2% to about 1% (w/w), about 0.2% to about 0.5% (w/w), about 0.3% to about 2% (w/w), about 0.3% to about 1.5% (w/w), about 0.3% to about 1% (w/w), about 0.3% to about 0.5% (w/w), about 0.4% to about 2% (w/w), about 0.4% to about 1.5% (w/w), about 0.4% to about 1% (w/w), about 0.4% to about 0.5% (w/w), about 0.5% to about 2% (w/w), about 0.5% to about 1.5% (w/w), about 0.5% to about 1% (w/w), about 0.6% to about 2% (w/w), about 0.6% to about 1.5% (w/w), about 0.6% to about 1% (w/w), about 0.7% to about 2% (w/w), about 0.7% to about 1.5% (w/w), about 0.7% to about 1% (w/w), about 0.8%, to about 2% (w/w), about 0.8% to about 1.5% (w/w), about 0.8% to about 1% (w/w), about 0.9% to about 2% (w/w), about 0.9% to about 1.5% (w/w), about 0.9% to about 1% (w/w), about 1% to about 2% (w/w), about 1% to about 1.5% (w/w), about 1.5% to about 2% (w/w), or about 0.01%, 0.05%, 0.1%, 0.2%, 0.3%, 0.4%, 0.5%, 0.6%, 0.7%, 0.8%, 0.9%, 1%, 1.25%, 1.5%, 1.75%, or 2% (w/w) based on the total weight of the hair care composition. In certain embodiments, the preservative is selected from the group consisting of phenoxyethanol, ethyl glycerin, capryl glycol, sorbic acid and combinations thereof. In certain embodiments, the hair care composition further comprises phenoxyethanol. In certain embodiments, the hair care composition further comprises ethyl glycerin. In certain embodiments, the hair care composition further comprises phenoxyethanol and ethyl glycerin. In certain embodiments, the hair care composition further comprises phenoxyethanol, capryl glycol and sorbic acid (e.g. Optiphen Plus). In certain embodiments, phenoxyethanol is present in an amount from about 0.01% to about 2% (w/w) based on the total weight of the hair care composition. In certain embodiments, phenoxyethanol is present in an amount from about 0.01% to about 2% (w/w), about 0.01% to about 1.5% (w/w), about 0.01% to about 1% (w/w), about 0.01% to about 0.5% (w/w), about 0.1% to about 2% (w/w), about 0.1% to about 1.5% (w/w), about 0.1% to about 1% (w/w), about 0.1% to about 0.5% (w/w), about 0.2% to about 2% (w/w), about 0.2% to about 1.5% (w/w), about 0.2% to about 1% (w/w), about 0.2% to about 0.5% (w/w), about 0.3% to about 2% (w/w), about 0.3% to about 1.5% (w/w), about 0.3% to about 1% (w/w), about 0.3% to about 0.5% (w/w), about 0.4% to about 2% (w/w), about 0.4% to about 1.5% (w/w), abut 0.4% to about 1% (w/w), about 0.4% to about 0.5% (w/w), about 0.5% to about 2% (w/w), about 0.5% to about 1.5% (w/w), about 0.5% to about 1% (w/w), about 1% to about 2% (w/w), about 1% to about 1.5% (w/w), about 1.5% to about 2% (w/w), or about 0.01%, 0.05%, 0.1%, 0.2%, 0.3%, 0.4%, 0.5%, 0.6%, 0.7%, 0.8%, 0.9%, 1%, 1.25%, 1.5%, 1.75%, or 2% (w/w) based on the total weight of the hair care composition. In certain embodiments, ethyl glycerin is present in an amount from about 0.01% to about 2% (w/w) based on the total weight of the hair care composition. In certain embodiments, ethyl glycerin is present in an amount from about 0.01% to about 2% (w/w), about 0.01% to about 1.5% (w/w), about 0.01% to about 1% (w/w), about 0.01% to about 0.5% (w/w), about 0.1% to about 2% (w/w), about 0.1% to about 1.5% (w/w), about 0.1% to about 1% (w/w), about 0.1% to about 0.5% (w/w), about 0.2% to about 2% (w/w), about 0.2% to about 1.5% (w/w), about 0.2% to about 1% (w/w), about 0.2% to about 0.5% (w/w), about 0.3% to about 2% (w/w), about 0.3% to about 1.5% (w/w), about 0.3% to about 1% (w/w), about 0.3% to about 0.5% (w/w), about 0.4% to about 2% (w/w), about 0.4% to about 1.5% (w/w), about 0.4% to about 1% (w/w), about 0.4% to about 0.5% (w/w), about 0.5% to about 2% (w/w), about 0.5% to about 1.5% (w/w), about 0.5% to about 1% (w/w), about 1% to about 2% (w/w), about 1% to about 1.5% (w/w), about 1.5% to about 2% (w/w), or about 0.01%, 0.05%, 0.1%, 0.2%, 0.3%, 0.4%, 0.5%, 0.6%, 0.7%, 0.8%, 0.9%, 1%, 1.25%, 1.5%, 1.75%, or 2% (w/w) based on the total weight of the hair care composition. In certain embodiments, phenoxyethanol, capryl glycol and sorbic acid are present in an amount from about 0.5% to about 1.5% (w/w) based on the total weight of the hair care composition. In certain embodiments, phenoxyethanol, capryl glycol and sorbic acid are present in an amount from about 0.5% to about 1.5% (w/w), 0.5% to about 1% (w/w), about 1% to about 1.5% (w/w), or about 0.5%, 0.75%, 1%, 1.25%, or 1.5% (w/w) based on the total weight of the hair care composition.

In certain embodiments, the hair care composition further comprises a fragrance. In certain embodiments, the hair care composition further comprises a phthalate-free fragrance. In certain embodiments, the hair care composition is fragrance-free.

In an embodiment, a hair care composition comprises water in an amount from about 40% to about 70% (w/w), teasel root extract in an amount from about 0.5% to about 20% (w/w), ashwagandha leaf extract in an amount from about 0.5% to about 15% (w/w), rosemary leaf extract in an amount from about 0.5% to about 15% (w/w), mulberry leaf extract in an amount from about 0.5% to about 15% (w/w), malva leaf extract in an amount from about 0.5% to about 15% (w/w), Echinacea purpurea root extract in an amount from about 0.5% to about 15% (w/w), glycerin in an amount from about 2% to about 15% (w/w), propanediol in an amount from about 1% to about 10% (w/w), PEG 40 hydrogenated castor oil in an amount from about 0.5% to about 2% (w/w), PEG-7 glyceryl cocoate in an amount from about 0.6% to about 3% (w/w), Ceteareth-25 in an amount from about 15% to about 25% (w/w), tetrasodium glutamate diacetate in an amount from about 0.25% to about 0.75% (w/w), maltodextrin in an amount from about 5% to about 25% (w/w), a preservative and a phthalate-free fragrance, wherein the amounts are based on the total weight of the hair care composition.

In an embodiment, a hair care composition comprises water in an amount from about 30% to about 70% (w/w), teasel root extract in an amount from about 15% to about 20% (w/w), ashwagandha leaf extract in an amount from about 0.5% to about 15% (w/w), rosemary leaf extract in an amount from about 0.5% to about 15% (w/w), mulberry leaf extract in an amount from about 0.5% to about 15% (w/w), malva leaf extract in an amount from about 0.5% to about 15% (w/w), Echinacea purpurea root extract in an amount from about 0.5% to about 15% (w/w), glycerin in an amount from about 2% to about 15% (w/w), propanediol in an amount from about 1% to about 10% (w/w), PEG 40 hydrogenated castor oil in an amount from about 0.5% to about 2% (w/w), PEG-7 glyceryl cocoate in an amount from about 0.6% to about 3% (w/w), Ceteareth-25 in an amount from about 19% to about 25% (w/w), tetrasodium glutamate diacetate in an amount from about 0.25% to about 0.75% (w/w), maltodextrin in an amount from about 20% to about 25% (w/w), a preservative and a phthalate-free fragrance, wherein the amounts are based on the total weight of the hair care composition.

In an embodiment, a hair care composition comprises water in an amount from about 30% to about 70% (w/w), teasel root extract in an amount from about 14% to about 18% (w/w), ashwagandha leaf extract in an amount from about 0.5% to about 15% (w/w), rosemary leaf extract in an amount from about 0.5% to about 15% (w/w), mulberry leaf extract in an amount from about 0.5% to about 15% (w/w), malva leaf extract in an amount from about 0.5% to about 15% (w/w), Echinacea purpurea root extract in an amount from about 0.5% to about 15% (w/w), glycerin in an amount from about 2% to about 15% (w/w), propanediol in an amount from about 1% to about 10% (w/w), PEG 40 hydrogenated castor oil in an amount from about 0.5% to about 2% (w/w), PEG-7 glyceryl cocoate in an amount from about 0.6% to about 3% (w/w), Ceteareth-25 in an amount from about 14% to about 18% (w/w), tetrasodium glutamate diacetate in an amount from about 0.25% to about 0.75% (w/w), maltodextrin in an amount from about 15% to about 19% (w/w), a preservative and a phthalate-free fragrance, wherein the amounts are based on the total weight of the hair care composition.

In an embodiment, a hair care composition comprises water in an amount from about 30% to about 70% (w/w), teasel root extract in an amount from about 12% to about 15% (w/w), ashwagandha leaf extract in an amount from about 0.5% to about 15% (w/w), rosemary leaf extract in an amount from about 0.5% to about 15% (w/w), mulberry leaf extract in an amount from about 0.5% to about 15% (w/w), malva leaf extract in an amount from about 0.5% to about 15% (w/w), Echinacea purpurea root extract in an amount from about 0.5% to about 15% (w/w), glycerin in an amount from about 2% to about 15% (w/w), propanediol in an amount from about 1% to about 10% (w/w), PEG 40 hydrogenated castor oil in an amount from about 0.5% to about 2% (w/w), PEG-7 glyceryl cocoate in an amount from about 0.6% to about 3% (w/w), Ceteareth-25 in an amount from about 12% to about 16% (w/w), tetrasodium glutamate diacetate in an amount from about 0.25% to about 0.75% (w/w), maltodextrin in an amount from about 10% to about 14% (w/w), a preservative and a phthalate-free fragrance, wherein the amounts are based on the total weight of the hair care composition.

In certain embodiments, a hair care composition of the present disclosure can be formulated as a salve or pomade. In certain embodiments, the hair care composition of the present disclosure can be formulated as a hair oil, hair cream, hair-dressing, hair lotion, styling gel, pomatum, ointment, balm or salve.

In certain embodiments, hair care compositions of the present disclosure can: (i) reduce hair breakage; (ii) stimulate the hair follicles; (iii) soothe the scalp; (iv) increase length retention; (iv) decrease dryness; and/or (vi) provide sculpting to the hair. Subsequently, the salve and pomade styling treatment hair compositions described herein could include any combination of the above embodiments that work to reduce antimicrobial activity of the scalp, aid in length retention, reduce breakage, and decrease dryness all while promoting the overall health of the hair and scalp.

In certain embodiments, a hair care composition of the present disclosure can not include petroleum jelly or mineral oil. In certain embodiments, a hair care composition of the present disclosure can not include petroleum jelly. In certain embodiments, a hair care composition of the present disclosure can not include mineral oil.

### Methods of Use

In some embodiments, a method for treating postpartum shedding can comprise applying a hair care composition of the present disclosure to hair.

In some embodiments, a method for styling or sculpting hair comprises applying a hair care composition of the present disclosure to hair.

In some embodiments, a method for reducing and/or preventing hair breakage comprises applying a hair care composition of the present disclosure to hair.

In some embodiments, a method for stimulating hair follicles and/or hair growth comprises applying a hair care composition of the present disclosure to hair and/or a scap.

In some embodiments, a method for preventing hair loss comprises applying a hair care composition of the present disclosure to hair and/or a scalp.

In some embodiments, a method for soothing the scalp comprises applying a hair care composition of the present disclosure to a scalp.

In some embodiments, a method for increasing length retention of hair comprises applying a hair care composition of the present disclosure to hair.

In some embodiments, a method for decreasing dryness of hair and/or hydrating hair comprises applying a hair care composition of the present disclosure to hair.

In some embodiments, a method for relieving eczema and/or psoriasis comprises applying a hair care composition of the present disclosure to a calp.

In some embodiments, a method of hair edging follicles comprises applying a hair care composition of the present disclosure to hair.

In some embodiments, a method for improving blood circulation to the scalp comprises applying a hair care composition of the present disclosure to a scalp.

In some embodiments, a method of preventing dandruff comprises applying a hair care composition of the present disclosure to hair and/or a scalp.

In some embodiments, a method of reducing antimicrobial activity of the scalp comprises applying a hair care composition of the present disclosure to a scalp. In certain embodiments, the method results in length retention and/or reduction in shedding.

In certain embodiments, an amount of the hair care composition applied is an “effective amount.” An “effective amount” can be understood as an amount sufficient to bring about a desired effect and/or treatment outcome.

In certain embodiments, the hair is textured hair. In certain embodiments, the hair is type 2, type 3 or type 4 textured hair. In certain embodiments, the hair is type 2 textured hair. In certain embodiments, the hair is type 3 textured hair. In certain embodiments, the hair is type 4 textured hair.

In certain embodiments, the hair and/or scalp is on a subject. In certain embodiments, the subject is a human.

### Methods of Extraction and Methods of Preparation

In some embodiments, a method for preparing a plant extract that can be used in a hair care composition of the present disclosure can include the steps of combining a plant material with extractant containing glycerin and propanediol and allowing the plant material to be extracted for a period of time to yield a plant extract. In certain embodiments, the extractant can comprise up to about 52% (w/w) of glycerin and propanediol based on the total weight of the extractant. By way of example, but not limitation, the amount of glycerin can be between about 0.1% (w/w) and 51.9% (w/w) and the amount of propanediol can be between about 0.1% (w/w) and about 51.9% (w/w) based on the total weight of the extractant. In certain embodiments, the amount of glycerin can be about 0.1%, 0.5%, 1%, 2%, 3%, 4%, 5%, 6%, 7%, 8%, 9%, 10%, 15%, 20%, 25%, 30%, 35%, 40%, 45%, 50%, 51.9% (w/w) or any range or value therebetween based on the total weight of the extractant. In certain embodiments, the amount of propanediol can be about 0.1%, 0.5%, 1%, 2%, 3%, 4%, 5%, 6%, 7%, 8%, 9%, 10%, 15%, 20%, 25%, 30%, 35%, 40%, 45%, 50%, 51.9% (w/w) or any range or value therebetween based on the total weight of the extractant. In certain embodiments, the extractant comprises 45% (w/w) glycerin and 7% (w/w) propanediol. In certain embodiments, the propanediol is 1,3-propanediol. In certain embodiments, the extractant further comprises water. In certain embodiments, the extractant further comprises sodium lactate. In certain embodiments, the extractant further comprises water and sodium lactate. In certain embodiments, the extractant comprises 45% (w/w) glycerin, 7% (w/w) propanediol, 40% (w/w) water, and 2% (w/w) sodium lactate. In certain embodiments, the amount of plant material used can be from about 0.5 g to about 10 g. In certain embodiments, the amount of plant material can be about 1 g, 2 g, 3 g, 4 g, 5 g, 6 g, 7 g, 8 g, 9 g, or 10 g. In certain embodiments, the plant material is a plant material corresponding to any of the plant extracts disclosed herein or can be any plant material. In certain embodiments, the plant material can be a seed, root, leaf, stem or any other plant part. In certain embodiments, the plant material is a plant material as described herein. Any suitable period of time can be used.

In certain embodiments, the period of time can be from about 23 to about 36 hours. By way of example, but not limitation, the period of time can be from about 23 to about 36 hours, about 23 to about 32 hours, about 23 to about 30 hours, about 23 to about 28 hours, about 23 to about 25 hours, about 24 to about 36 hours, about 24 to about 32 hours, about 24 to about 30 hours, about 24 to about 28 hours, about 24 to about 25 hours, about 25 to about 36 hours, about 25 to about 32 hours, about 25 to about 30 hours, about 25 to about 28 hours, about 28 to about 36 hours, about 28 to about 32 hours, about 28 to about 30 hours, about 30 to about 36 hours, about 30 to about 32 hours, about 32 to about 36 hours, or about 23, 24, 25,26, 27, 28, 29, 30, 31, 31, 33, 34, 35, or 36 hours. In certain embodiments, the period of time is about 24 hours.

In certain embodiments, the method for preparing the plant extract can further comprise a step of heating the plant material with extractant for a second period of time. In certain embodiments, the second period of time can be from about 30 minutes to about 1 hour. In certain embodiments, the second period of time can be about 30 minutes. In certain embodiments, the second period of time can be about 1 hour. In certain embodiments, the step of heating the plant material with extractant can be performed at a temperature of between about 60° C. and about 80° C., i.e. heating the mixture and holding it for the second period of time at that temperature. In certain embodiments, the plant material and extractant can be heated to about 75° C. By way of example, but not limitation, the extract blend can be heated to about 60° C. to about 80° C., about 60° C. to about 75° C., about 60° C. to about 70° C., about 60° C. to about 65° C., about 65° C. to about 80° C., about 65° C. to about 75° C., about 65° C. to about 70° C., about 70° C. to about 80° C., about 70° C. to about 75° C., about 75° C. to about 80° C., about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

It should be understood that a plant extract of the disclosure can be produced as a single extract or multiple extracts. In certain embodiments, the resulting plant extract can comprise about 95% glycerin and about 5% propanediol. In certain embodiments, the plant extract can comprise glycerin and propanediol. In certain embodiment, the propanediol is 1,3-propanediol.

In some embodiments, a method for preparing a hair care composition can include the steps of combining a teasel root extract and the one or more additional extracts, e.g. an ashwagandha leaf extract, a rosemary leaf extract, a mulberry leaf extract, a malva leaf extract, and an Echinacea purpurea root extract, the water, the humectant and, optionally, the stabilizer to yield an extract formulation; combining the extract formulation with a formulation base; mixing the extract formulation and formulation base to yield a mixed extract and formulation base; combining the mixed extract formulation and formulation base with additional ingredients; and allowing the mixed extract formulation, formulation base and additional ingredients to solidify to yield the hair care composition. In certain embodiments, the humectant comprises glycerin and propanediol. In certain embodiments, the stabilizer comprises tetrasodium glutamate diacetate. In certain embodiments, the formulation base comprises the shaper or a portion thereof and the emollient. In certain embodiments, the shaper comprises Ceteareth-25. In certain embodiments, the emollient comprises PEG 40 hydrogenated castor oil and PEG-7 glyceryl cocoate. In certain embodiments, the additional ingredients comprise the remaining portion of the shaper. In certain embodiments, the remaining portion of the shaper can comprise maltodextrin. In certain embodiments, the additional ingredients further comprise the preservative and the fragrance. In certain embodiments, the preservative comprises ethyl glycerin and phenoxyethanol. In certain embodiments, the fragrance comprises a pthalate-free fragrance. In certain embodiments, the hair care composition is a salve or pomade. In certain embodiments, the amounts of materials can be sufficient to yield the desired final amount in the hair care composition.

In certain embodiments, the extract blend can be heated. In certain embodiments, the extract blend can be heated to about 60° C. to about 80° C. In certain embodiments, the extract blend can be heated to about 75° C. By way of example, but not limitation, the extract blend can be heated to about 60° C. to about 80° C., about 60° C. to about 75° C., about 60° C. to about 70° C., about 60° C. to about 65° C., about 65° C. to about 80° C., about 65° C. to about 75° C., about 65° C. to about 70° C., about 70° C. to about 80° C., about 70° C. to about 75° C., about 75° C. to about 80° C., about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In certain embodiments, the formulation base can be heated. In certain embodiments, the formulation base can be heated to about 60° C. to about 80° C. In certain embodiments, the formulation base can be heated to about 75° C. By way of example, but not limitation, the formulation base can be heated to about 60° C. to about 80° C., about 60° C. to about 75° C., about 60° C. to about 70° C., about 60° C. to about 65° C., about 65° C. to about 80° C., about 65° C. to about 75° C., about 65° C. to about 70° C., about 70° C. to about 80° C., about 70° C. to about 75° C., about 75° C. to about 80° C., about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In certain embodiments, the heating step(s) can be from about 30 minutes to about 24 hours. In certain embodiments the heating step can be about 1 hour.

In certain embodiments, after combining the heated components in the hair care composition, the hair care composition can be allowed to cool and/or finish for up to 24 hours (inclusive of the heating steps). It should be understood that the hair care composition can be allowed to sit longer, including during shelf storage, but that a cooling and finishing period can be included after heating for the hair care composition to solidify.

In certain embodiments, the mixing of the extract formulation and formulation base can be performed by agitation. In certain embodiments, the agitation can be by using a blender.

It should be understood that the extracts used to produce the hair care composition can be produced by any suitable method. Preferably, the extracts are produced from an extraction using propanediol and glycerin. Alternatively, the extracts can be produced using standard methods such as a glycerin extraction. It was surprisingly found that an extraction using propanediol was more effective in extracting plant material and functioned more as an emollient, producing a less sticky extract.

It should be further understood that where an extract comprising glycerin and propanediol is used in the hair care composition, the final hair care composition can comprise about 0.01% to 1% of glycerin and propanediol from the extracts.

In certain embodiments, the method for preparing the hair care composition can further comprise preparing the teasel root extract, the ashwagandha leaf extract, the rosemary leaf extract, the mulberry leaf extract, the malva leaf extract, and the Echinacea purpurea root extract. In certain embodiments, the step of preparing each of the extracts can comprise hydrating plant material with 45% (w/w) glcyerin, 7% (w/w) propanediol, 40% (w/w) water and 2% (w/w) sodium lactate under conditions sufficient to produce a raw extract; and straining the raw extract to yield the plant extract. In certain embodiments, the teasel root extract can be prepared by hydrating teasel root with 45% glycerin, 7% propanediol, 40% water and 2% sodium lactate under conditions sufficient to produce a raw teasel root extract; and straining the raw teasel root extract to yield the teasel root extract. In certain embodiments, the ashwagandha leaf extract can be prepared by hydrating ashwagandha leaves with 45% glycerin, 7% propanediol, 40% water and 2% sodium lactate under conditions sufficient to produce a raw ashwagandha leaf extract; and straining the raw ashwagandha leaf extract to yield the ashwagandha leaf extract. In certain embodiments, the rosemary leaf extract can be prepared by hydrating rosemary leaves with 45% glycerin, 7% propanediol, 40% water and 2% sodium lactate under conditions sufficient to produce a raw rosemary leaf extract; and straining the raw rosemary leaf extract to yield the rosemary leaf extract. In certain embodiments, the mulberry leaf extract can be prepared by hydrating mulberry leaves with 45% glycerin, 7% propanediol, 40% water and 2% sodium lactate under conditions sufficient to produce a raw mulberry leaf extract; and straining the raw mulberry leaf extract to yield the mulberry leaf extract. In certain embodiments, the malva leaf extract can be prepared by hydrating malva leaves with 45% glycerin, 7% propanediol, 40% water and 2% sodium lactate under conditions sufficient to produce a raw malva leaf extract; and straining the raw malva leaf extract to yield the malva leaf extract. In certain embodiments, the Echinacea purpurea root extract can be prepared by hydrating Echinacea purpurea root with 45% glycerin, 7% propanediol, 40% water and 2% sodium lactate under conditions sufficient to produce a raw Echinacea purpurea root extract; and straining the raw Echinacea purpurea root extract to yield the Echinacea purpurea root extract.

In an embodiment, a method for making a hair care composition of the present disclosure as a salve and pomade styling treatment hair composition is provided. The method for making this salve and pomade styling treatment includes the steps of separately hydrating ashwagandha leaves, rosemary leaves, mulberry leaves, malva leaves, teasel root, Echinacea purpurea root with glycerin, propanediol, water, and sodium lactate to create the botanical and antimicrobial extracts for the hair composition. Next, the hydrated botanical and antimicrobial extracts for the hair composition are each strained and stored in their own containers. Next, water, glycerin, propanediol, tetrasodium glutamate diacetate, and the extracts are heating in a vessel to 78° C. In an alternative embodiment, apricot extract and fenugreek seed extract are added in the previous step. Next, ceteareth-25, PEG 40 hydrogenated castor oil, and PEG 7 glyceryl cocoate are combined in a vessel and heated to 78° C. The ingredients in both heated vessels are then combined together and mixed with an immersion blender until they are uniformed. Lastly, maltodextrin, ethyl glycerin, phenoxyethanol, and a phthalate-free fragrance are added to the composition and poured into jars to solidify. In certain aspects, the glycerin for the extraction is present in an amount of about 45%, the propanediol for the extraction can be present in an amount of about 7%, the water for the extraction can be present in an amount of about 40%, and the sodium lactate for the extraction can be present in an amount of about 2%.

### EXAMPLES

### Example 1: Preparation of Hair Care Composition

Teasel roots, ashwagandha leaves, rosemary leaves, mulberry leaves, malva leaves and Echinacea purpurea roots were each separately hydrated for 23 hours at room temperature after heating to 75° C. for 1 hour with 45% glycerin, 7%1,3-propanediol, 40% water, and 2% sodium lactate to generate extracts of each plant material. Each of the extracts was the strained to remove solids.

Each of the extracts was combined in addition to water, glycerin, propanediol and tetrasodium glutamate diacetate in a vessel and heated to 78° C. Separately, Ceteareth-25, PEG 40 hydrogenated castor oil and PEG-7 glyceryl cocoate were combined a vessel and heated to 78° C. The contents of each vessel were then combined and agitated with a blender to ensure uniform distribution of the ingredients.

Maltodextrin, ethyl glycerin, phenoxyethanol and a phthalate-free fragrance were then added to the mixture and the resulting hair care composition was poured into jars and allowed to solidify.

An exemplary formulation that was prepared is provided in the Table below:

| Ingredient | Amount (w/w) |
|---|---|
| Water | 44.2% |
| Ashwagandha Leaf Extract | 0.5% |
| Rosemary Leaf Extract | 0.5% |
| Mulberry Leaf Extract | 0.5% |
| Malva Leaf Extract | 0.5% |
| Teasel Root Extract | 0.5% |
| Echinacea purpurea Root Extract | 0.5% |
| Glycerin | 7% |
| Propanediol | 5% |
| Ceteareth-25 | 29.5% |
| PEG 40 Hydrogenated Castor Oil | 1% |
| PEG-7 Glyceryl Cocoate | 1% |
| Maltodextrin | 8% |
| Tetrasodium glutamate diacetate | 0.5% |
| Phenoxyethanol and ethyl glycerin | 0.8% |

### Example 2: Use of Hair Care Composition

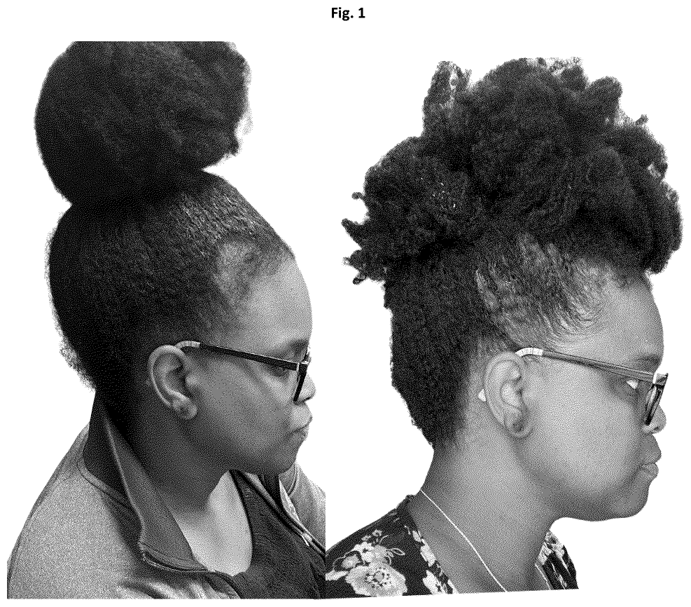

A human subject suffering from postpartum shedding applied the hair care composition of claim 1 to her hair, including the affected area. Briefly, a small dab of product about the size of a fore finger was applied. FIG. 1 shows a picture before application of the hair care composition (left) and a picture 4 months after application of the hair care composition (right) demonstrating the effectiveness of the hair care composition in restoring hair growth.

All publications, patents and patent applications cited in this specification are herein incorporated by reference as if each individual publication or patent application were specifically and individually indicated to be incorporated by reference.

The disclosure set forth above may encompass multiple distinct embodiments with independent utility. Although each of these embodiments has been disclosed in its preferred form(s), the specific embodiments thereof as disclosed and illustrated herein are not to be considered in a limiting sense, because numerous variations are possible. The subject matter of embodiments disclosed herein includes all novel and nonobvious combinations and subcombinations of the various elements, features, functions, and/or properties disclosed herein. The following claims particularly point out certain combinations and subcombinations regarded as novel and nonobvious. Embodiments embodied in other combinations and subcombinations of features, functions, elements, and/or properties may be claimed in this application, in applications claiming priority from this application, or in related applications. Such claims, whether directed to a different embodiment or to the same embodiment, and whether broader, narrower, equal, or different in scope in comparison to the original claims, also are regarded as included within the subject matter of the present disclosure.

What is claimed is:

1. A hair care composition, comprising:
from about 0.5% to about 20% (w/w) teasel root extract; and
one or more additional extracts selected from the group consisting of ashwagandha leaf extract, rosemary leaf extract, mulberry leaf extract, malva leaf extract, Echinacea purpurea root extract, and combinations thereof,
wherein each of the one or more additional extracts is present in an amount from about 0.5% to about 15% (w/w) based on the total weight of the composition.

2. The hair care composition of claim 1, wherein the one or more additional extracts comprise a combination of ashwagandha leaf extract, rosemary leaf extract, mulberry leaf extract, malva leaf extract, and Echinacea purpurea root extract.

3. The hair care composition of claim 1, further comprising a shaper (holder), an emollient and a humectant.

4. The hair care composition of claim 2, wherein the shaper is selected from the group consisting of maltodextrin, Ceteareth-25, and combinations thereof; the emollient is selected from the group consisting of PEG-7 glyceryl cocoate, PEG 40 hydrogenated castor oil and combinations thereof; and the humectant is selected from the group consisting of glycerin, propanediol, and combinations thereof.

5. The hair care composition of claim 1, further comprising glycerin, propanediol, PEG 40 hydrogenated castor oil, PEG-7 glyceryl cocoate, Ceteareth-25 and maltodextrin.

6. The hair care composition of claim 5, wherein the glycerin is present in an amount from about 2% to about 15% (w/w) based on the total weight of the hair care composition, the propanediol is present in an amount from about 2% to about 15% (w/w) based on the total weight of the hair care composition, the PEG 40 hydrogenated castor oil is present in an amount from about 0.5% to about 2% (w/w) based on the total weight of the hair care composition, the PEG-7 glyceryl cocoate is present in an amount from about 0.6% to about 3% (w/w) based on the total weight of the hair care composition, the Ceteareth-25 is present in an amount from about 10% to about 30% (w/w) based on the total weight of the hair care composition, and the maltodextrin is present in an amount from about 5% to about 25% (w/w) based on the total weight of the hair care composition.

7. The hair care composition of claim 5, wherein the glycerin is present in an amount from about 5% to about 10% (w/w) based on the total weight of the hair care composition, the propanediol is present in an amount from about 2% to about 10% (w/w) based on the total weight of the hair care composition, the PEG 40 hydrogenated castor oil is present in an amount from about 0.5% to about 2% (w/w) based on the total weight of the hair care composition, the PEG-7 glyceryl cocoate is present in an amount from about 0.6% to about 3% (w/w) based on the total weight of the hair care composition, the Ceteareth-25 is present in an amount from about 25% to about 30% (w/w) based on the total weight of the hair care composition, and the maltodextrin is present in an amount from about 5% to about 15% (w/w) based on the total weight of the hair care composition.

8. The hair care composition of claim 5, further comprising a stabilizer, wherein the stabilizer comprises tetrasodium glutamate diacetate.

9. The hair care composition of claim 8, wherein the stabilizer is present in an amount from about 0.25% to about 0.75% (w/w) based on the total weight of the hair care composition.

10. The hair care composition of claim 5, further comprises a preservative, wherein the preservative is a combination of phenoxyethanol and ethyl glycerin.

11. The hair care composition of claim 10, wherein the preservative is present in an amount from about 0.01% to about 2% (w/w) based on the total weight of the hair care composition.

12. The hair care composition of claim 1, further comprising apricot extract in an amount from about 0.5% to about 5% (w/w) based on the total weight of the hair care composition and fenugreek seed extract in an amount from about 0.5% to about 5% (w/w) based on the total weight of the hair care composition.

13. The hair care composition of claim 5, further comprising a phthalate-free fragrance.

14. The hair care composition of claim 5, wherein the hair care composition is fragrance-free.

15. A method for styling or sculpting hair, comprising:
applying the composition of claim 5 to hair.

16. The method of claim 15, wherein the hair is textured hair, and wherein the textured hair is type 2 textured hair, type 3 textured hair, or type 4 textured hair.

17. A method for producing a plant extract, comprising:
combining a plant material with extractant containing glycerin and propanediol and allowing the plant material to be extracted for a period of time to yield a plant extract.

18. The method of claim 17, wherein the extractant comprises about 45% by weight glycerin and about 7% by weight propanediol and, optionally, about 40% water by weight and 2% sodium lactate by weight, based on the total weight of the extractant.

19. The method of claim 17, wherein the period of time is about 24 hours.

20. A method for producing a hair care composition, comprising:
combining a teasel root extract and optionally, an ashwagandha leaf extract, a rosemary leaf extract, a mulberry leaf extract, a malva leaf extract, and an Echinacea purpurea root extract, water, an humectant and, optionally, a stabilizer to yield an extract formulation;
combining the extract formulation with a formulation base;
mixing the extract formulation and formulation base to yield a mixed extract and formulation base;
combining the mixed extract formulation and formulation base with additional ingredients; and
allowing the mixed extract formulation, formulation base and additional ingredients to solidify to yield the hair care composition.
